(19) United States
(12) Patent Application Publication
(10) Pub. No.: US 2020/0046477 A1
(43) Pub. Date: Feb. 13, 2020

(54) KIT AND METHOD FOR TRACKING A HUMAN JAW

(71) Applicant: Claronav Inc., North York (CA)

(72) Inventor: Doron Dekel, Toronto (CA)

(21) Appl. No.: 16/344,396

(22) PCT Filed: Nov. 2, 2017
(86) PCT No.: PCT/CA2017/051302

§ 371 Date: Apr. 24, 2019

(51) Int. Cl. A61C 19/045; A61B 17/86; A61C 1/08

(52) U.S. Cl. CPC A61C 19/045; A61B 2034/2055; A61C 1/084; A61B 17/8605

(57) ABSTRACT

A kit and method for tracking a human jaw are provided. The kit and method can involve a bone screw and an extension arm. The bone screw has a threaded portion, a head portion and a longitudinal axis. The threaded portion can have a thread diameter of less than or equal to about 2.5 mm and can be inserted into a bone of the human jaw within 45° of the occlusal plane. The head portion protrudes out of the bone of the human jaw when the threaded portion is inserted into the bone. The extension arm can be rigidly secured to the head portion of the bone screw to maintain a fixed spatial mapping between the head portion of the bone screw and an optically detectable marking provided on the extension arm. When secured to the head portion of the bone screw, the extension arm can be positioned such that the optically detectable marking is detectable by an optical tracking system positioned outside the mouth containing the jaw bone.

### CROSS REFERENCE TO RELATED APPLICATIONS

This application claims the benefit U.S. Provisional Application No. 62/417,405 filed Nov. 4, 2016 and entitled Jaw Attachment for Dental Navigation, the entirety of which is incorporated herein by reference.

### FIELD

The embodiments described herein relate to the field of oral surgery, and in particular to a kit and method for tracking a human jaw.

### BACKGROUND

PCT patent application PCT/CA2015/051287, titled “Appliance for Dental Navigation”, which is incorporated herein by reference, teaches optical tracking of a jaw bone using a retainer appliance. However, for at least some patients, there remains a need for alternative ways of providing optical tracking of a jaw bone.

### SUMMARY

In accordance with an aspect of an embodiment of the present invention, there is provided a kit for tracking a human jaw comprising: i) a bone screw having a threaded portion, a head portion and a longitudinal axis, wherein the threaded portion has a thread diameter of less than or equal to about 2.5 mm and is insertable into a bone of the human jaw, and the head portion protrudes out of the bone of the human jaw when the threaded portion is inserted into the bone; and ii) an extension arm comprising a) a head-receiving portion for receiving the head portion of the bone screw, b) an optically detectable marking trackable by an optical tracking system to uniquely determine a pose of the optically detectable marking in a reference coordinate system, and c) a fastener for detachably and rigidly securing the head portion of the bone screw to the head-receiving portion to maintain a fixed spatial mapping between the head portion of the bone screw and the optically detectable marking of the extension arm, wherein, when secured to the head portion of the bone screw, the extension arm is positionable such that the optically detectable marking is detectable by an optical tracking system positioned outside the mouth containing the jaw bone.

In some embodiments, the extension arm comprises an optically trackable attachment and a receiving portion. The optically trackable attachment comprises the optically detectable marking and is detachably and rigidly attachable to the receiving portion of the extension arm to maintain the fixed spatial mapping between the head portion of the bone screw and the optically detectable marking of the extension arm.

In some embodiments, the extension arm comprises at least one optically trackable attachment engaging surface, and the optically trackable attachment comprises a corresponding connecting region. When the extension arm is detachably and rigidly attached to the optically trackable attachment, the at least one engaging surface is aligned with the corresponding connecting region of the optically trackable attachment to define a pose of the optically trackable attachment in relation to the extension arm according to the fixed spatial mapping for the optically trackable attachment.

In some embodiments, the extension arm further comprises an unweakened portion for withstanding and a weakened portion for breaking when a force acting on the extension arm generates a torque tending to rotate the bone screw about an axis of rotation substantially orthogonal to the longitudinal axis of the bone screw, and the torque exceeds a torque threshold that would break the bone screw. The force acts at a point spaced from the bone screw by a lengthwise distance along the extension arm. The lengthwise distance defines a moment arm, such that the weakened portion is between the bone screw and the point spaced from the bone screw.

In some embodiments, the unweakened portion of the extension arm comprises at least one bendable portion separate from the weakened portion for bending in response to the force acting on the extension arm such that the point at which the force acts, spaced apart from the bone screw by the lengthwise distance along the extension arm, is movable in a direction substantially parallel to the longitudinal axis of the bone screw by at least 2 mm without breaking the weakened portion.

In some embodiments, the head-receiving portion of the extension arm comprises a cavity for receiving the head portion; and, the head portion of the bone screw comprises at least one head surface. The cavity of the head-receiving portion of the extension arm comprises at least one cavity surface for aligning with the at least one head surface to impede rotation of the head portion within the cavity about the longitudinal axis of the bone screw when the head portion is rigidly secured within the head-receiving portion by the fastener.

In some embodiments, the head portion of the bone screw comprises at least one recess for engaging the fastener of the extension arm to impede relative movement between the head portion of the bone screw and the head-receiving portion of the extension arm.

In some embodiments, the head portion of the bone screw comprises i) a base, comprising the at least one head surface; and ii) a neck attached to the base for engaging the fastener of the extension arm. The neck is rotatable within the cavity to align the at least one head surface of the base with the at least one cavity surface of the head-receiving portion. The base is not rotatable about the longitudinal axis of the bone screw within the cavity when the fastener is engaged with the neck to hold the base within the cavity and the at least one head surface of the base against the at least one cavity surface.

In some embodiments, the head portion of the bone screw further comprises a tip attached to the neck for restraining the fastener of the extension arm between the base and the tip when the fastener is engaged with the neck. The tip and the neck are rotatable within the cavity to align the at least one head surface of the base with the at least one cavity surface of the head-receiving portion. The head-receiving portion of the extension arm is not moveable along the longitudinal axis of the bone screw when the fastener is engaged with the neck.

In some embodiments, the neck portion comprises at least one recess for engaging the fastener of the extension arm to impede relative movement between the head portion of the bone screw and the head-receiving portion of the extension arm.

In some embodiments, the thread diameter of the bone screw is less than about 2.3 mm.

In accordance with an aspect of an embodiment of the present invention, there is provided a method for tracking changes in a pose of a jaw bone displaceable in a reference coordinate system, the jaw bone defining an occlusal plane. The method involves: i) inserting a bone screw in an insertion direction into a bone of the jaw bone, the insertion direction being within 45° of the occlusal plane, the bone screw comprising an insertable portion for inserting into the jaw bone, and a head portion for protruding from the jaw bone; ii) detachably and rigidly attaching a head-receiving portion of an extension arm to a head portion of the bone screw to maintain a fixed spatial mapping between the head portion of the bone screw and the extension arm; iii) providing an optically detectable marking on the extension arm such that the optically detectable marking is detectable by an optical tracking system located outside the mouth containing the jaw bone; iv) detecting the optically detectable marking using the optical tracking system; and v) operating the optical tracking system to measure the changes in the pose of the jaw bone within the reference coordinate system based on the changes in the pose of the optically detectable marking.

In some embodiments, inserting the bone screw involves i) providing the bone screw having a thread diameter of less than or equal to about 2.5 mm; ii) drilling a pilot hole having a hole diameter less than the thread diameter of the bone screw in one of the upper jaw bone and the lower jaw bone along the insertion direction; and iii) inserting the bone screw into the pilot hole.

In some embodiments, the described method involves: operating a computer processor, in electronic communication with the optical tracking system, to use the measured pose changes to guide positioning surgical tools and implants relative to the jaw bone.

In some embodiments, inserting the bone screw in the insertion direction into the bone of the jaw bone involves inserting the bone screw in an insertion direction within 30° of the occlusal plane.

In some embodiments, providing the optically detectable marking on the extension arm involves rigidly attaching an optically trackable attachment to the extension arm, the optically trackable attachment comprising the optically detectable marking.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a side perspective view of an assembled kit for tracking a jaw bone, in accordance with an embodiment;

FIG. 2 is a side view of a bone screw of the kit of FIG. 1;

FIG. 3A is a side perspective view of the bone screw of FIG. 2, inserted into a jaw bone at a substantially vertical orientation (for a patient whose head is upright);

FIG. 3B is a side perspective view of the bone screw of FIG. 2, inserted into the jaw bone at a substantially horizontal orientation (for a patient whose head is upright);

FIG. 4A is a partial side perspective view of an extension arm to be used for a bone screw that can be inserted into the jaw bone at a substantially vertical orientation in accordance with an embodiment of the invention;

FIG. 4B is a partial side perspective view of an extension arm to be used for a bone screw that can be inserted into the jaw bone at a substantially horizontal orientation in accordance with an embodiment of the invention;

FIG. 5A is a side perspective view of an extension arm secured to a bone screw inserted into the jaw bone at a substantially vertical orientation in accordance with an embodiment of the invention;

FIG. 5B is a side perspective view of an extension arm secured to a bone screw inserted into the jaw bone at a substantially horizontal orientation in accordance with an embodiment of the invention;

FIG. 6 is an enlarged side perspective of a head-receiving portion of the extension arm shown in FIG. 4A; and

FIG. 7 is a sectional view of an extension arm secured to a bone screw inserted into the jaw bone at a substantially vertical orientation in accordance with an aspect of an embodiment of the present invention.

FIG. 8 is the sectional view of FIG. 7 showing additional aspects of embodiments of the invention; and

FIG. 9 is a flow chart of a method for tracking a human jaw in accordance with an embodiment.

### DETAILED DESCRIPTION OF EXAMPLE EMBODIMENTS

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

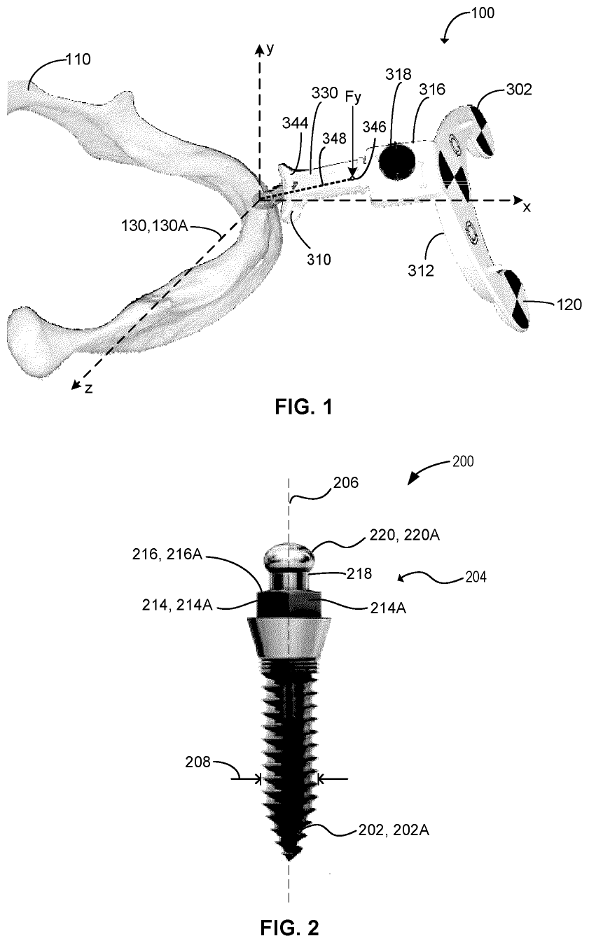

Referring to FIG. 1, illustrated therein is an assembled kit 100 for tracking a jaw bone 110 that includes a bone screw 200 (shown in FIG. 2) and an extension arm 300 securable to the bone screw 200. The bone screw 200 can be inserted into the jaw bone 110. As shown in FIG. 1, the jaw bone 110 may be that of a human patient (not shown). The extension arm 300 may include an optically detectable marking 302 trackable by an optical tracking system (not shown) to uniquely determine a pose 120 of the optically detectable marking 302 in a reference coordinate system 130. In the example shown in FIG. 1, the reference coordinate system 130 can be a Cartesian coordinate system 130A. Alternatively, other reference coordinate systems can be used, including, without limitation, polar coordinate systems such as spherical or cylindrical coordinate systems. When secured to the bone screw 200, the extension arm 300 can be positioned such that the optically detectable marking 302 can be detected by the optical tracking system positioned outside a mouth (not shown) containing the jaw bone 110.

The kit 100 for tracking the jaw bone 110 can be used for oral surgery concerning a planned restoration, such as dental implants. An image (not shown) of the jaw bone 110 can be produced, for example, by a computerized tomography (CT) scan and registered to the jaw bone 110 using one of several registration methods well known in the art of surgical navigation system design. The optically detectable marking 302 can be configured to be trackable by the optical tracking system to determine the pose 120 of the optically detectable marking 302. The pose 120 of the optically detectable marking 302 can be tracked to track the position of the jaw bone 110, to enable adjusting an image-bone registration mapping such that its accuracy is maintained during and following movements of the jaw bone 110 in the reference coordinate system 130.

The optical tracking system can be in electrical communication with a computer processor (not shown) of a surgical navigation system (not shown). The computer processor can be operable to use measured pose changes to guide positioning surgical tools and implants relative to the jaw bone 110. For example, the computer processor of the surgical navigation system can map a surgical plan prepared in a coordinate system of the image, or the tip of a tool being tracked separately (not shown) to the reference coordinate system 130 of the jaw bone 110, established by the optically detectable marking 302 even when the jaw bone 110 moves. The accuracy of the registration can be verified through a verification process in which the oral surgeon, by touching a prominent landmark (not shown) in the mouth of the patient with a drill-tip (not shown), can compare the actual location of the drill-tip with the location shown on the image derived by the CT scan. Optionally, the oral surgeon can insert a second bone screw (not shown) away from the bone screw 200 prior to, for example, the CT scan, to be used as the prominent landmark in the verification process.

Referring still to FIG. 1, the kit 100 for tracking a jaw bone 110 can be configured to rigidly and detachably secure the bone screw 200 to the extension arm 300 thereby maintaining a fixed spatial mapping (not shown) between a head portion 204 of the bone screw 200 (shown in FIG. 2) and the optically detectable marking 302 of the extension arm 300.

While the embodiments described herein show the bone screw 200 of the kit 100 for tracking a jaw bone 110 inserted into a lower jaw bone (mandible) 110, the bone screw 200 can also be inserted into an upper jaw (maxilla) (not shown) with the kit 100 for tracking a jaw bone 110 functioning in an equivalent manner. The choice of whether to insert the bone screw 200 into the lower jaw bone 110 or the upper jaw bone may depend on the location of the planned restoration.

Referring to FIG. 2, illustrated therein is the bone screw 200 including an insertable portion 202, the head portion 204 and a longitudinal axis 206. For example, the insertable portion 202 may be a threaded portion 202A having a thread diameter 208 of less than or equal to about 3.0-mm. FIG. 2 shows an embodiment of the bone screw 200 with the threaded portion 202A having a 2.2-mm thread diameter 208. In an alternative embodiment (not shown), the threaded portion 202A may have a 2.5-mm thread diameter 208. The threaded portion 202A can be inserted into the jaw bone 110 (shown in FIG. 1) by any suitable means. For example, a pilot hole (not shown) can first be made in the jaw bone 110 using a pilot drill (not shown). A socket wrench (not shown) can then be used to drive the bone screw 200 into the pilot hole. The pilot drill can be sized to drill a pilot hole having a diameter smaller than the thread diameter 208 of the bone screw 200. For example, for the bone screw 200 with the 2.2-mm thread diameter 208, the pilot drill may have a 1.5-mm diameter. Alternatively, for the bone screw 200 with the 2.5-mm thread diameter 208, the pilot drill may have a 1.8-mm diameter. The pilot drill can be used to drill the pilot hole in either the upper jaw bone or the lower jaw bone 110 depending on the location of the planned restoration. The head portion 204 protrudes out of the jaw bone 110 when the insertable portion 202 is inserted in the jaw bone 110.

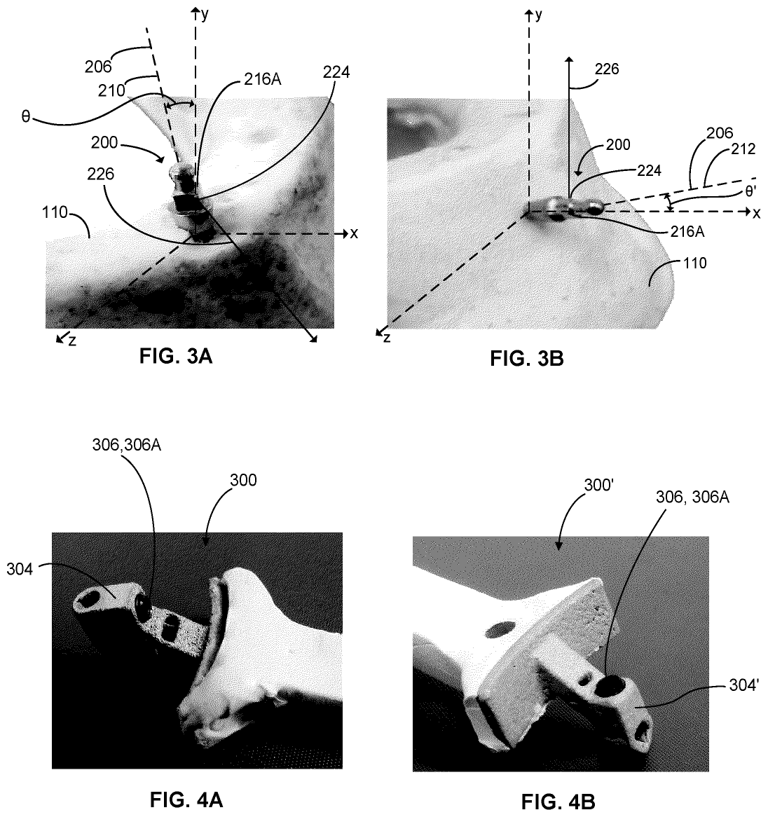

Reference is now made to FIGS. 3A and 3B which show the bone screw 200 inserted into the jaw bone 110. In both FIGS. 3A and 3B, an occlusal plane can correspond to the x-z plane of the Cartesian coordinate system. A labial plane, substantially perpendicular the occlusal plane, can correspond to the y-z plane of the Cartesian coordinate system. In FIG. 3A, the bone screw 200 can be inserted into the jaw bone 110 such that the longitudinal axis 206 of the bone screw 200 may be at an insertion angle, θ, relative to the labial plane. For example, the insertion angle θ can be within 0 to 45° of the labial plane. In an alternative embodiment, the insertion angle θ can be within 0 to 30° of the labial plane. In FIG. 3B, the bone screw 200 can be inserted into the jaw bone 110 such that the longitudinal axis 206 of the bone screw 200 may be at an insertion angle, θ′, relative to the occlusal plane. For example, the insertion angle θ′ can be within 0 to 45° of the occlusal plane. In an alternative embodiment, the insertion angle θ′ can be within 0 to 30° of the occlusal plane. The range of insertion angles θ and θ′ permissible to insert the bone screw 200 into the jaw bone 110 can allow the oral surgeon to select an insertion angle θ and θ′ that can best allow the bone screw 200 to be surrounded by an area of dense bone to reduce the risk of premature release of the bone screw 200 or damage to the bone.

Referring specifically to the example shown in FIG. 3A, the bone screw 200 can be inserted into the jaw bone 110 in which the longitudinal axis 206 of bone screw 200 has a substantially vertical orientation 210 for a patient whose head is upright (in which pose, the substantially vertically oriented bone screw 200 will be substantially perpendicular to the occlusal plane). Referring specifically to the example shown in FIG. 3B, the bone screw 200 can be inserted into the jaw bone 110 in which the longitudinal axis 206 of the bone screw 200 has a substantially horizontal orientation 212 for a patient whose head is upright (in which pose, the substantially horizontally oriented bone screw 200 will be substantially parallel to the occlusal plane). In general, a substantially horizontal orientation 212 can be preferable since the bone screw 200 interferes less with the wearing of dentures and with bone ridge reshaping operations during surgery. It can be preferable to select an area of dense bone in which to insert the bone screw 200 to reduce the risk of premature release of bone screw 200.

Reference is now made to FIGS. 4A and 4B. Referring specifically to FIG. 4A, illustrated therein is a partial view of an extension arm such as the extension arm 300 shown in FIG. 1. The extension arm 300 can include a head-receiving portion 304 for receiving the head portion 204 of the bone screw 200 (shown in FIG. 3A), a fastener 306 for detachably and rigidly securing the head portion 204 of the bone screw 200 (shown in FIG. 3A) to the head-receiving portion 304, and the optically detectable marking 302 (shown in FIG. 1). The optically detectable marking 302 may be trackable by an optical tracking system to uniquely determine the pose 120 of the optically detectable marking 302 in the reference coordinate system 130 (shown in FIG. 1).

Referring still to FIG. 4A, the fastener 306 may detachably and rigidly secure the head portion 204 of the bone screw 200 (shown in FIG. 3A) to the head-receiving portion 304 of the extension arm 300 to maintain the fixed spatial mapping between the head portion 204 of the bone screw 200 and the optically detectable marking 302 of the extension arm 300. As shown in FIG. 4A, the fastener 306 may be a threaded set screw 306A that can be tightened and loosened using an Allen-type key (not shown) or a screw driver (not shown). The head-receiving portion 304 of the extension arm 300 may contain a threaded fastener hole 308 (shown in FIG. 6) for receiving the threaded screw 306A. If the extension arm 300 needs to be removed from the bone screw 200, the fastener 306 can be released using the Allen-type key or the screw driver. When secured, the fastener 306 can provide the fixed spatial mapping between the head portion 204 of the bone screw 200 and the optically detachable marking 302 of the extension arm 300.

Referring now to FIG. 4B, illustrated therein is a partial view of an extension arm 300′. The extension arm 300′ shows an alternative embodiment of the extension arm 300 shown in FIG. 4A. The extension arm 300′ differs from extension arm 300 in the configuration of the head-receiving portion 304 and the head receiving portion 304′. When the extension arm 300 and 300′ is secured to the head portion 204 of the bone screw 200, the extension arm 300 and 300′ may be positionable such that the optically detectable marking 302 is detectable by the optical tracking system positioned outside the mouth (not shown) of the jaw bone 110 (shown in FIG. 1). In this way, either the extension arm 300 or the extension arm 300′ can be used depending on whether the bone screw 200 has been inserted into the jaw bone 110 at the substantially vertical orientation 210 or the substantially horizontal orientation 212 (shown in FIGS. 3A and 3B, respectively) so as to allow the optically detectable marking 302 to be positionable outside the mouth. If the bone screw 200 has been inserted into the jaw bone 110 at the substantially vertical position 210 (Shown in FIG. 3A), the extension arm 300 may be used. Alternatively, if the bone screw 200 has been inserted into the jaw bone 110 at the substantially horizontal orientation 212 (Shown in FIG. 3B), the extension arm 300′ may be used.

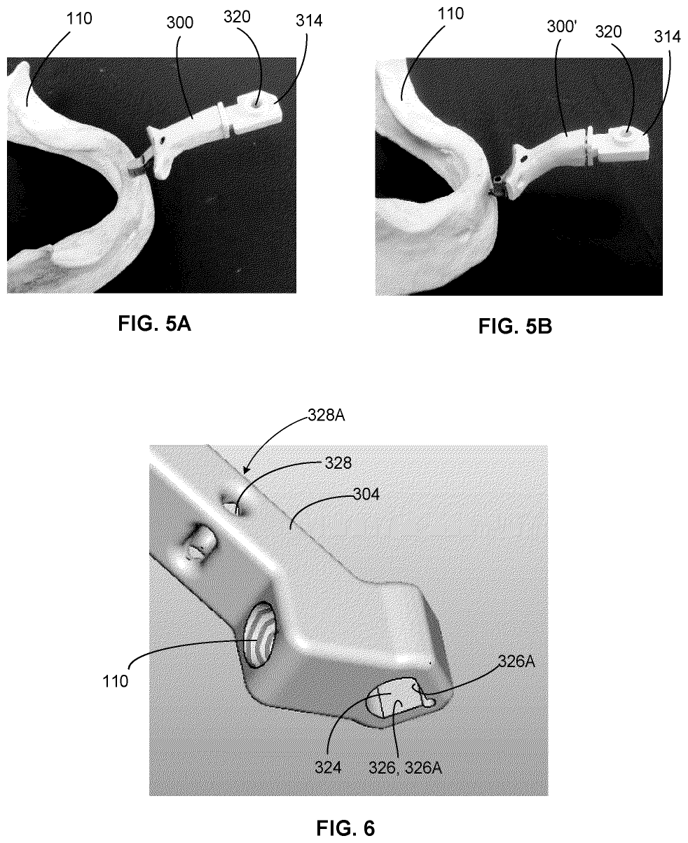

Reference is now made to FIGS. 5A and 5B. FIG. 5A shows the extension arm 300 secured to the bone screw 200 inserted into the jaw bone 110 at the substantially vertical orientation 210 (shown in FIG. 3A). FIG. 5B shows the extension arm 300′ secured to the bone screw 200 inserted into the jaw bone 110 at the substantially horizontal orientation 212 (shown in FIG. 3B). As shown in FIGS. 5A and 5B, the receiving-head portion 304 of the extension arm 300 and 300′ can be oriented to allow the extension arm 300 and 300′ to extend out of the mouth when secured to the bone screw 200.

Referring back to FIG. 1, the extension arm 300 is shown having a receiving portion 310 to which an optically trackable attachment 312 can be detachably and rigidly attached. The optically trackable attachment 312 can include the optically detectable marking 302. The extension arm 300 may further include at least one optically trackable attachment engaging surface 314 (shown in FIG. 5A). The optically trackable attachment 312 may include a connection region 316 that corresponds to (aligns or mates with) the at least one optically trackable attachment engaging surface 314. In this way, when the extension arm 300 can be detachably and rigidly attached to the optically trackable attachment 312, the at least one engagement surface 314 can be aligned with the corresponding connecting region 316 of the optically trackable attachment 312 in relation to the extension arm 300 to maintain a fixed spatial mapping between the extension arm 300 and the optically trackable attachment 312. The fixed spatial mapping between the extension arm 300 and the optically trackable attachment 312 can, in turn, maintain the fixed spatial mapping between the head portion 204 of the bone screw 200 and the optically detectable marking 302 of the extension arm 300.

Referring still to FIG. 1, a fastener 318 can be used to detachably and rigidly attach the receiving portion 310 to the optically trackable attachment 312. As shown in FIG. 1, the fastener 318 can be a thumb-screw 318A that may be tightened and loosened by hand. In a preferred embodiment, the at least one optically detectable attachment engaging surface 314 of the extension arm 300 may contain a threaded thru-hole 320 (shown on FIG. 5A) and the connecting region 316 of the optically trackable attachment 312 may contain a threaded fastener hole (not shown) as to allow the fastener 318 to pass through the threaded thru-hole 320 and be received by the threaded fastener hole. In an alternative embodiment (not shown), any suitable means to detachably and rigidly secured the at least one optically detectable attachment engagement surface 314 to the connecting region 316 can be used. In still further embodiments (not shown), the receiving portion 310 may be integrally formed with the optically trackable attachment 312. When secured, the fastener 318 can fix the optically trackable attachment 312 including the optically detectable marking 302 in relation to the extension arm 300 thereby maintaining the fixed spatial mapping between the head portion 204 of the bone screw 200 and the optically detectable marking 302 of the extension arm 300.

Referring again to FIG. 2, the head portion 204 of bone screw 200 may include at least one head surface 214. In accordance with an embodiment, the head portion 204 can be configured to include a base 216, a neck 218 attached to the base 216, and a tip 220 attached to the neck 218. The base 216 can include the at least one head surface 214. In a preferred embodiment, the base 216 may include four head surfaces 214A configured as a squad head 216A. The square head 216A can allow the bone screw 200 to be inserted into the jaw bone 110 using a square socket wrench (not shown). In alternative embodiments (not shown), the square head 216A may be an oval head with one head surface, a triangle head with three head surfaces, a hexagonal head with six head surfaces, or another shape that is not continuously rotationally symmetric. As described below, the at least one head surface 214 can prevent the extension arm 300 and 300′ (shown in FIGS. 5A and 5B, respectively) from rotating relative to the bone screw 200 about the longitudinal axis 206 of the bone screw 200.

Referring now to FIG. 6, illustrated therein is an enlarged view of the head-receiving portion 304 of the extension arm 300. The head-receiving portion 304 can include a cavity 324 for receiving the head portion 204 of the bone screw 200 (shown in FIG. 3A). The cavity 324 can include at least one cavity surface 326 for aligning with the at least one head surface 214 of the head portion 204 of the bone screw 200 (shown in FIG. 3A). When the cavity 324 of the head-receiving portion 304 of the extension arm 300 receives the head portion 204 of the bone screw 200 (Shown in FIG. 3A), the at least one cavity surface 326 can align with the at least one head surface 214 of the head portion 204 of the bone screw 200 (shown in FIG. 3A). This alignment can impede relative rotation between the head portion 204 of the bone screw 200 and the head-receiving portion 304 of the extension arm 300 about the longitudinal axis 206 of the bone screw 200. By impeding relative rotation between the head portion 204 of the bone screw 200 and the head-receiving portion 304 of the extension arm, the optically trackable attachment 312 can be maintained in the fixed spatial mapping relative to the head portion 204 of bone screw 200 as the extension arm 300 is rigidly secured to the head portion 204 of the bone screw 200, and the optically trackable attachment 312 is rigidly secured to the extension arm 300.

Referring still to FIG. 6, in a preferred embodiment, the cavity 324 may include two cavity surfaces 326A such that the two cavity surfaces 326A can align with two adjacent head surfaces of the four head surfaces 214A of the square head 216A (shown in FIG. 3A). As shown in FIGS. 3A and 3B, the bone screw 200 can be inserted into the jaw bone 110 such that a corner 224 of the square head 216A points in a desired direction 226 of the extension arm 300 and 300′ (shown in FIGS. 5A and 5B).

Referring back to FIG. 2, the tip 220 is shown as a ball tip 220A, having a diameter equal to a length of the at least one head surface 214. In an alternative embodiment (not shown), the tip 220 may be a square tip with the same dimensions as the square head 216A. The tip 220 can be sized to be equal to or smaller than the base 216 as to allow the cavity 324 of head-receiving portion 304 of the extension arm 300 (shown in FIG. 6) to receive the head portion 204 of the bone screw 200. When head-receiving portion 304 of the extension arm 300 has received the head portion 204 of the bone screw 200, the neck 218 can be engaged by the fastener 306 of the extension arm 300 (shown in FIG. 4A).

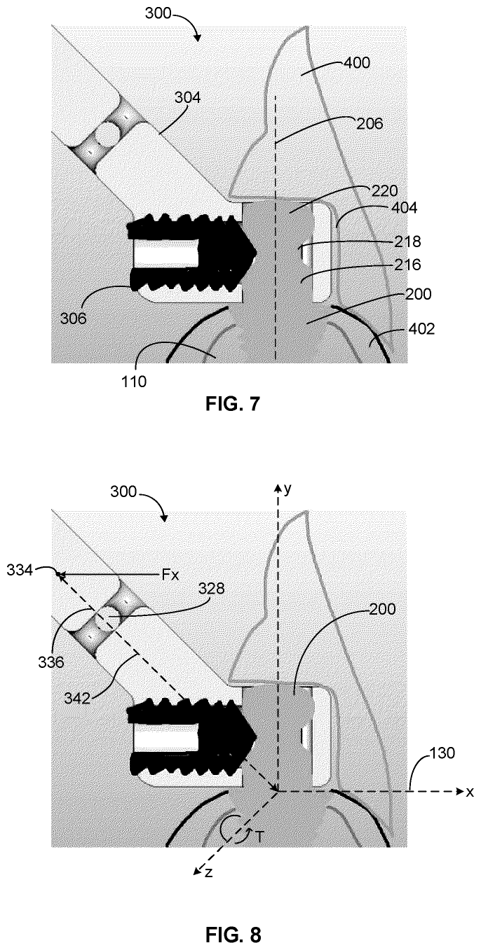

Referring to FIG. 7, illustrated therein is a sectional view of the extension arm 300 when detachably and rigidly secured to the bone screw 200 in the substantially vertical orientation 210. As shown in FIG. 7, when the fastener 306 is engaged with the neck 218, the fastener 306 can be restrained between the tip 220 and the base 216 of the head portion 204. In this way, this engagement can impede the head-receiving portion 304 of extension arm 300 from moving vertically along the longitudinal axis 206 of the bone screw 200. By impeding vertical movement of the head-receiving portion 304 about the longitudinal axis 206 of the bone screw 200, the fixed spatial mapping can be maintained between the head portion 204 of the bone screw 200 and the optically trackable attachment 312 of extension arm 300 (shown in FIG. 1).

Referring again to FIG. 2, the head portion 204 of the bone screw 200 may further include at least one recess (not shown) that can engage with the fastener 306 of the extension arm 300 (shown in FIGS. 4A and 4B). In a preferred embodiment, the at least one recess can be located on the neck 218 of the head portion 204. When the fastener 306 is tightened into the at least one recess, relative movement between the head portion 204 of the bone screw 200 and the head-receiving portion 304 (shown in FIGS. 4A and 4B) of the extension arm 300 may be impeded. In an alternative embodiment (not shown), a plurality of recesses can be provided, wherein each recess can be placed to correspond to locations where the fastener 306 may be engaged.

Referring again to FIG. 6, the extension arm 300 may further include a weakened portion 328 and an unweakened portion 330 (shown in FIG. 1). In accordance with an embodiment, the weakened portion 328 can be formed on the head-receiving portion 304 of the extension arm 300. In a preferred embodiment, the weakened portion 328 can be formed by removing material from head-receiving portion 304, leaving a section of corner posts 328A. As will be described below, the weakened portion 328 can provide a safety feature that may protect the jaw bone 110 from breaking or the bone screw 200 from breaking.

Referring to FIG. 8, illustrated therein is a sectional view of the extension arm 300 when detachably and rigidly secured to the bone screw 200 in the substantially vertical orientation 210 (shown in FIG. 3A). The following description also describes the extension arm 300′ when detachably and rigidly secured to the bone screw in the substantially horizontal orientation 212. The extension arm 300 may be accidentally hit during oral surgery thereby applying a force F to the extension arm 300 (designated Fx in FIG. 8 because in the example shown the force is parallel to the x-axis in the Cartesian coordinate system shown). In the example shown in FIG. 8, force F acts at a point 334 spaced from the bone screw 200 by a lengthwise distance 336 along the extension arm 300. The lengthwise distance 336 defines a moment arm 342, such that the weakened portion 328 is between the bone screw 200 and the point 334 spaced from the bone screw 200. The force F generates a torque, T, about the z-axis. In the example shown in FIG. 8, where the longitudinal axis 206 of the bone screw 200 is parallel to the y-axis in the Cartesian coordinate system, the weakened portion 328 can be configured to break when torque exceeds a safe toque threshold and tends to rotate the bone screw 200 about any axis of rotation in the x-z plane. In general, the weakened portion 328 can be configured to break when the torque exceeds the safe toque threshold and tends to rotate the bone screw 200 about any axis of rotation substantially orthogonal to the longitudinal axis 206 of the bone screw 200.

Breaking of the extension arm 300 can prevent unsafe torque from being transmitted to the bone screw 200 that could potentially injure the jaw bone 110 or break the bone screw 200. For example, the safe torque threshold to break the weakened portion 328 can set between 25 and 100 N·cm. In a preferred embodiment, the safe torque threshold can be set at 50 N·cm. In a still further embodiment, the safe torque threshold can be varied based on the strength of the bone screw 200, which, in turn, may depend on the thread diameter 208 of the bone screw 200 (other things equal, thicker bone screws may be stronger). The unweakened portion 330 may withstand torques exceeding the safe torque threshold without breaking.

Referring still to FIG. 8, the safety feature described above is less important for torque tending to rotate the bone screw 200 about the y-axis since the this torque can simply rotate the bone screw 200 about its longitudinal axis 206 instead of breaking the surrounding bone.

Referring again to FIG. 1, the unweakened portion 330 of the extension arm 300 may include at least one bendable portion 344 separate from the weakened portion 328 for bending in response to a force F (designated Fy in FIG. 1 because in the example shown the force is parallel to the y-axis in the Cartesian coordinate system 130A). For example, a point 346 at which the force F acts, spaced apart from the bone screw 200 by a lengthwise distance 348 along the extension arm 300, can move by at least 2 mm in a direction substantially parallel (not shown) to the longitudinal axis 206 of the bone screw 200 (shown in FIG. 3A) without breaking the weakened portion 328 (shown in FIG. 6). The at least one bendable portion 344 can absorb the force F by bending up to a bending limit (not shown) before the remainder of the force F is transmitted to the bone screw 200 as torque. In this way, the at least one bendable portion 344 can prevent the weakened portion 328 from breaking in the event the extension arm 300 secured to the optically trackable attachment 312 is accidentally hit during oral surgery by absorbing a portion, or all, of the hit. To preserve the fixed spatial mapping between the head portion 204 of the bone screw 200 and the optically detectable marking 302 of the extension arm 300, the at least one bendable portion 344 of the extension arm 300 can be biased to return to its original position, after bending to absorb the force F.

Referring again to FIG. 6, the head-receiving portion 304 of the extension arm 300 can be made, for example, from an aluminum alloy. The aluminum alloy can provide the minimum required strength for rigidly securing the head portion of bone screw 200. Unlike other metals, the aluminum alloy does not cause unacceptable beam hardening and “streak” image artifacts that degrade the scan image.

Referring again to FIG. 1, the receiving portion 310 and the optically trackable attachment 312 of the extension arm 300 can be made, for example, from a flexible thermoplastic material designed to bend thereby absorbing or minimizing the force F before it is transmitted to the bone screw 200 as torque.

Referring again to FIG. 7, illustrated therein is a sectional view of the extension arm 300 when detachably and rigidly secured to the bone screw 200. As shown, when the bone screw 200 is inserted into the jaw bone 110, the head portion 204 protrudes from the jaw bone 110 and gums 402. It can be advantageous to scan a patient (not shown) with a scan appliance 400, which can outline the contours of the planned restoration and gums 402 on a scan image (not shown). This can be accommodated by carving a cavity 404 in the scan appliance 400 in a region that would otherwise come in contact with the head portion 204 of the bone screw 200 protruding from the jaw bone 110 and the gums 402. The cavity 404 can enable proper seating of the scan appliance 400 on the gums 402. If the patient wears dentures (not shown) when the bone screw 200 is inserted into the jaw bone 110, a similar cavity (not shown) can be made in the dentures to accommodate the head portion 204 of the bone screw 200.

At the conclusion of oral surgery, the bone screw 200 may either be removed, or left in place if the bone screw 200 can be advantageously used to support the planned restoration.

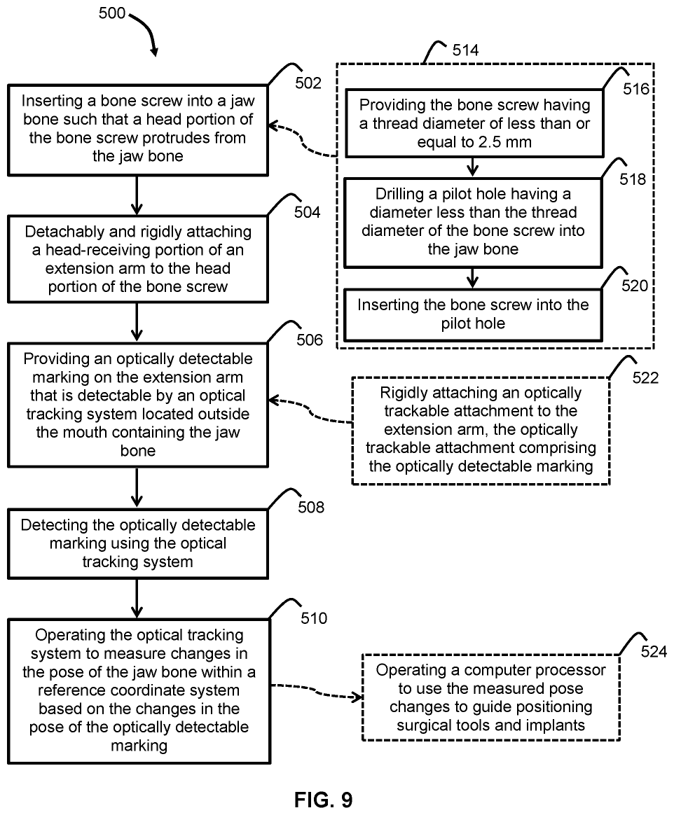

Referring to FIG. 9, illustrated therein is a method 500 for tracking changes in a pose of a jaw bone displaceable in a reference coordinate system, in accordance with an embodiment. The jaw bone may define the occlusal plane (shown in FIGS. 3A and 3B as the x-z plane). At 502, a bone screw can be inserted into the jaw bone at an insertion angle within 45° of the occlusal plane. For example, the bone screw can inserted at the insertion angle of 10° or less relative to the occlusal plane. Alternatively, for example, the bone screw can be inserted at the insertion angle of 30° or less relative to the occlusal plane. The bone screw can include an insertable portion that can be inserted into the jaw bone and a head portion that can protrude from the jaw bone when the bone screw is inserted into the jaw bone. At 504, a head-receiving portion of an extension arm can be detachably and rigidly attached to the head portion of the bone screw. The detachable and rigid attachment between the head-receiving portion of the extension arm and the head portion of the bone screw can maintain a fixed spatial mapping between the head portion of the bone screw and the extension arm. At 506, an optically detectable marking can be provided on the extension arm such that the optically detectable marking can be detectable by an optical tracking system located outside the mouth containing the jaw bone. At 508, the optically detectable marking can be detected using the optical tracking system. At 510, the optical tracking system can be operated to measure changes in the pose of the jaw bone in the reference coordinate system based on changes in the pose of the optically detectable marking.

Referring still to FIG. 9, illustrated therein is a method 514 for inserting a bone screw into the jaw bone at 502, in accordance with an embodiment. According to some aspects of some embodiments, as described, for example, at 516, the bone screw can have a thread diameter of less than or equal to about 2.5 mm. At 518, a pilot hole having a hole diameter less than the thread diameter of the bone screw can be drilled into one of an upper jaw bone and a lower jaw bone. At 520, the bone screw can be inserted into the pilot hole.

Referring still to FIG. 9, the method 500 can further include different ways of providing the optically detectable marking on the extension arm at 506, in accordance with an embodiment. That is, at 522, an optically trackable attachment can be rigidly attached to the extension arm. The optically trackable attachment can include the optically detectable marking.

Referring still to FIG. 9, the method 500 can further include, at 524, operating a computer processor in electronic communication with the optical tracking system. The computer processor can use the measured pose changes to guide positioning surgical tools and implants relative to the jaw bone.

While the above description provides examples of one or more apparatus, methods, or systems, it will be appreciated that other apparatus, methods, or systems may be within the scope of the claims as interpreted by one of skill in the art.
A kit for tracking a human jaw, the kit comprising:
a bone screw having a threaded portion, a head portion and a longitudinal axis, wherein the threaded portion has a thread diameter of less than or equal to about 2.5 mm and is insertable into a bone of the human jaw, and the head portion protrudes out of the bone of the human jaw when the threaded portion is inserted into the bone;
an extension arm comprising i) a head-receiving portion for receiving the head portion of the bone screw, ii) an optically detectable marking trackable by an optical tracking system to uniquely determine a pose of the optically detectable marking in a reference coordinate system, and iii) a fastener for detachably and rigidly securing the head portion of the bone screw to the head-receiving portion to maintain a fixed spatial mapping between the head portion of the bone screw and the optically detectable marking of the extension arm, wherein, when secured to the head portion of the bone screw, the extension arm is positionable such that the optically detectable marking is detectable by an optical tracking system positioned outside the mouth containing the jaw bone.

2. The kit as defined in claim 1 wherein the extension arm comprises an optically trackable attachment and a receiving portion, the optically trackable attachment,
comprising the optically detectable marking; and,
being detachably and rigidly attachable to the receiving portion of the extension arm to maintain the fixed spatial mapping between the head portion of the bone screw and the optically detectable marking of the extension arm.

3. The kit as defined in claim 2 wherein
the extension arm comprises at least one optically trackable attachment engaging surface, and
the optically trackable attachment comprises a corresponding connecting region, such that when the extension arm is detachably and rigidly attached to the optically trackable attachment, the at least one engaging surface is aligned with the corresponding connecting region of the optically trackable attachment to define a pose of the optically trackable attachment in relation to the extension arm according to the fixed spatial mapping for the optically trackable attachment.

4. The kit as defined in claim 1 wherein the extension arm further comprises an unweakened portion for withstanding and a weakened portion for breaking when a force acting on the extension arm generates a torque tending to rotate the bone screw about an axis of rotation substantially orthogonal to the longitudinal axis of the bone screw, and the torque exceeds a torque threshold that would break the bone screw, the force acting at a point spaced from the bone screw by a lengthwise distance along the extension arm, the lengthwise distance defining a moment arm, such that the weakened portion is between the bone screw and the point spaced from the bone screw.

5. The kit as defined in claim 4 wherein the unweakened portion of the extension arm comprises at least one bendable portion separate from the weakened portion for bending in response to the force acting on the extension arm such that the point at which the force acts, spaced apart from the bone screw by the lengthwise distance along the extension arm, is movable in a direction substantially parallel to the longitudinal axis of the bone screw by at least 2 mm without breaking the weakened portion.

6. The kit as defined in claim 1 wherein
the head-receiving portion of the extension arm comprises a cavity for receiving the head portion; and,
the head portion of the bone screw comprises at least one head surface and the cavity of the head-receiving portion of the extension arm comprises at least one cavity surface for aligning with the at least one head surface to impede rotation of the head portion within the cavity about the longitudinal axis of the bone screw when the head portion is rigidly secured within the head-receiving portion by the fastener.

7. The kit as defined in claim 1 wherein the head portion of the bone screw comprises at least one recess for engaging the fastener of the extension arm to impede relative movement between the head portion of the bone screw and the head-receiving portion of the extension arm.

8. The kit as defined in claim 6 wherein
the head portion of the bone screw comprises a base, comprising the at least one head surface, and a neck attached to the base for engaging the fastener of the extension arm;
the neck is rotatable within the cavity to align the at least one head surface of the base with the at least one cavity surface of the head-receiving portion; and
the base is not rotatable about the longitudinal axis of the bone screw within the cavity when the fastener is engaged with the neck to hold the base within the cavity and the at least one head surface of the base against the at least one cavity surface.

9. The kit as defined in claim 8 wherein
the head portion of the bone screw further comprises a tip attached to the neck for restraining the fastener of the extension arm between the base and the tip when the fastener is engaged with the neck;
the tip and the neck are rotatable within the cavity to align the at least one head surface of the base with the at least one cavity surface of the head-receiving portion; and
the head-receiving portion of the extension arm is not moveable along the longitudinal axis of the bone screw when the fastener is engaged with the neck.

10. The kit as defined in claim 9 wherein the neck portion comprises at least one recess for engaging the fastener of the extension arm to impede relative movement between the head portion of the bone screw and the head-receiving portion of the extension arm.

11. The kit as defined in claim 1 wherein the thread diameter is less than about 2. 3 mm.

12. A method for tracking changes in a pose of a jaw bone displaceable in a reference coordinate system, the jaw bone defining an occlusal plane, the method comprising:
inserting a bone screw in an insertion direction into a bone of the jaw bone, the insertion direction being within 45° of the occlusal plane, the bone screw comprising an insertable portion for inserting into the jaw bone, and a head portion for protruding from the jaw bone;
detachably and rigidly attaching a head-receiving portion of an extension arm to a head portion of the bone screw to maintain a fixed spatial mapping between the head portion of the bone screw and the extension arm;
providing an optically detectable marking on the extension arm such that the optically detectable marking is detectable by an optical tracking system located outside the mouth containing the jaw bone;
detecting the optically detectable marking using the optical tracking system; and
operating the optical tracking system to measure the changes in the pose of the jaw bone within the reference coordinate system based on the changes in the pose of the optically detectable marking.

13. The method as defined in claim 12 wherein
inserting the bone screw comprises
providing the bone screw having a thread diameter of less than or equal to about 2.5 mm;
drilling a pilot hole having a hole diameter less than the thread diameter of the bone screw in one of an upper jaw bone and a lower jaw bone along the insertion direction; and
inserting the bone screw into the pilot hole.

14. The method as defined in claim 12 further comprising
operating a computer processor, in electronic communication with the optical tracking system, to use the measured pose changes to guide positioning surgical tools and implants relative to the jaw bone.

15. The method as defined in claim 13 wherein the insertion direction is within 30° of the occlusal plane.

16. The method as defined in claim 12 wherein providing the optically detectable marking on the extension arm comprises rigidly attaching an optically trackable attachment to the extension arm, the optically trackable attachment comprising the optically detectable marking.
